(19) United States
(12) Patent Application Publication
(10) Pub. No.: US 2006/0153915 A1
(43) Pub. Date: Jul. 13, 2006

(54) Sustained-release preparations and method for producing the same

(75) Inventors: Jin Woo Park, Seoul (KR); Joon Ho Bae, Seoul (KR); Jung Ju Kim, Yongin-si (KR)

Correspondence Address: JONES DAY, 222 EAST 41ST ST, NEW YORK, NY, 10017, US

(73) Assignee: AMOREPACIFIC CORPORATION, Seoul (KR)

(21) Appl. No.: 10/538,695

(22) PCT Filed: Jan. 19, 2004
(86) PCT No.: PCT/KR04/00092

§ 371 Date: Jun. 10, 2005

(30) Foreign Application Priority Data
Jan. 23, 2003 (KR) 10-2003-0004521

(51) Int. Cl. A61K 31/485; A61K 9/26

(52) U.S. Cl. 424/469; 514/282

(57) ABSTRACT

The present invention relates to sustained-release preparations prepared by double granulation and methods for producing the same. The sustained-release preparation according to the present invention enables maintenance of effective blood concentration of drug for many hours via sustained release of the drug over 12 hours or more, and further its production is easy owing to convenience of process.

### TECHNICAL FIELD

The present invention relates to sustained-release preparations and method for producing the same.

### BACKGROUND ART

Sustained-release preparations are such pharmaceuticals as exhibit pharmacological effect over a prolonged time, unlike immediate-release preparations which exhibit the pharmacological effect immediately upon being taken. In particular, sustained-release analgesics can solve inconvenience of taking medicine during sleep in postoperative or cancer patients suffering from a pain of medium level or more or patients who have a serious migraine so that they cannot p to sleep. Lately, based on increased clinical understanding of pains, analgesics have been used for various chronic diseases, and sustained-release analgesics have been used widely for prevention of pain or for providing convenience to postoperative outpatients.

In general, in case there is no restriction to dissolution and absorption of a drug at the gastrointestinal tract, blood level of the drug is controlled by delaying its absorption via controlled-release of drug from pharmaceuticals. That is, in case of drug with high water solubility, drug-including pellet is coated with release-delaying layer, or matrix tablet is prepared by mixing with hydrophobic material, leading to control of the diffusion of drug dissolved within dosage form, thereby imparting sustained-release property. Typical sustained-release preparations include coated pellets, coated tablets and capsules, and drug release through such preparations depends on unique property such as selective destruction of coating layer or swelling of inner matrix.

In case of simple matrix tablet, use of drug with high water solubility is accompanied by problems, i.e. relatively large amount of hydrophobic release-delaying agent is needed, and the size of tablet as well increases in proportion to that. Therefore, recently, studies have been conducted to modify surface properties of drug at molecular level through application of solid dispersion. Particles of the solid dispersion system are prepared by applying heat to mixture of melting additives and drug or by using solvent that can dissolve two substances at the same time. That is, in case of slightly soluble drug, bioavailability is increased by raising solubility through improving wetting property of the drug by use of hydrophilic additives such as polyethyleneglycol or polyvinylalcohol, while in case of hydrophilic drug, sustained-release property is imparted by reducing wetting of drug through use of hydrophobic additives. As the solid dispersion method allows modification of surface property of drug at molecular level, it is advantageous, that is, maximum effect can be obtained by use of minimum amount of additives, and actual production is easy owing to simplicity of process.

As preparation process based on solid dispersion, melt-extrusion and melt-granulation can be enumerated, and the melt-granulation has been known as preparation technology for sustained release preparations. The melt-granulation is a method where granules are formed by applying physical action to a mixture of drug, at least one kind of binders and additives to allow melted binders to adhere to the surface of drug particles. Detailed explanation thereof is as follows. Drug, at least one kind of binders and additives are subjected to physical mixing, energy is added until the binders or additives are melted. Then, this is cooled to prepare solid mass, this is pulverized to desirable size of pellets, the pellets were filled into capsule or mixed with additives and compressed to prepare sustained-release tablets. Preparation method for tramadol-including sustained-release preparations based on said technology was already disclosed in U.S. Pat. No. 5,591,452. On the other hand, melt-extrusion is similar to melt-granulation, yet differs in that processes of melting, extrusion, cooling and pulverization are carried out sequentially. Preparing process for drug-including sustained-release pellet by said technology is disclosed in WO 93/15753.

Sustained-release analgesics developed so far as once- or twice-a-day preparations are largely divided into matrix tablet using hydrophobic substance and pellets coated with release-delaying layer. U.S. Pat. No. 5,849,240, U.S. Pat. No. 5,891,471, U.S. Pat. No. 6,162,467 and U.S. Pat. No. 5,965,163 disclose a method in which sustained-release granules are prepared by melt granulation, and then prepared into tablet or capsule type. In addition, U.S. Pat. No. 6,261,599, U.S. Pat. No. 6,290,990 and U.S. Pat. No. 6,335,033 describe methods where sustained-release pellets are prepared by melt extrusion, and then prepared into tablet form. Additionally, U.S. Pat. No. 6,254,887 and U.S. Pat. No. 6,306,438 disclose methods other than the melt granulation and melt extrusion for preparing sustained-release pellets. That is, a method where inert beads were coated with drug layer, and then with sustained-release coating layer, or matrix pellets were prepared by use of binders such as wax and then coated with sustained-releasing layer, and a method where drug was dispersed in melted hydrophobic polymer and sprayed to prepare pellets, and a method of coating with melted wax for matrix granules including hydrophobic polymer and drug

According to said preparation methods, as drug surface can be covered with hydrophobic substances at molecular level, release-delaying can be effectively induced by use of just small amount of hydrophobic additive, and the process is simple. However, majority of the hydrophobic additives used in melt granulation and melt extrusion has property of wax, thus the surface of particles prepared by cooling after melting becomes to exhibit adhesion toward another surface. Therefore, problems occur in actual production, i.e. reduced flow of particles at hopper, severe adhesion to punch or die at the time of tablet compression and increased resistance at the time of removing tablet from tablet machine. Such adhesion problem can be covered to some degree by adding lubricants, yet the masking power is limited, thus the amount of hydrophobic additives is to be limited. Lubricant is generally used in 0.1 to 5%, at most, to the weight of granules. In case of using excessive amount of lubricants, release rate reduces, capping and laminating phenomena occur during tablet process, while phenomenon such as chipping and picking occurs in case of deficiency.

U.S. Pat. No. 5,955,104, U.S. Pat. No. 5,968,551, U.S. Pat. No. 6,159,501, U.S. Pat. No. 6,143,322 and PCT/EP1997/03934 disclose methods for preparing sustained release pellets as multi unit dosage form where inert beads were coated with drug layer, then with coating layer comprising alkyl cellulose and acrylic polymer. The prepared pellets were filled into capsules, and effective blood level of opiate analgesic was observed to maintain over 24 hours. In particular, U.S. Pat. No. 6,159,501 discloses that release rate can be controlled by mixing immediate-releasing uncoated pellets and sustained-releasing pellets and by filling into a capsule. On the other hand, U.S. Pat. No. 6,103,261 and U.S. Pat. No. 6,249,195 disclose a method for preparing sustained-release pellets to obtain analgesic effect over 24 hrs, in which matrix pellet comprising gun, alkylcellulose, acryl resin and drug was coated with acrylic polymer and ethyl cellulose. However, this method includes inconvenience, i.e. necessity of at least two times of coating and combination procedure of particles for later controlling drug release and content, and exhibits problems that in case of preparations requiring large content, volume of total particles is to be increased and further sustained-releasing property is to be reduced compared to compressed tablet due to increase in drug release area.

The present invention was conceived to resolve the problems of the conventional techniques, and its object lies in minimizing the amount of hydrophobic additives for imparting sustained-releasing property, and eliminating adhesion phenomenon of granules occurring during the tablet preparation, thereby allowing the production of tablet to be easy.

The present invention relates to sustained-release preparations and method for producing the same.

More specifically, the present invention relates to sustained-release preparations characterized by being prepared from double granules which are obtained by primary granulation of drug according to melt granulation using hydrophobic release-delaying additives, and then by secondary granulation of the obtained granules according to wet granulation using hydrophobic wet-granulation material.

It is preferred that said sustained-release preparations comprise 0.5 to 80% by weight of drug, 10 to 65% by weight of hydrophobic release-delaying additive, 1 to 35% by weight of hydrophobic wet-granulation material.

Said drug is not specifically limited, and for example, analgesic can be used. As an analgesic, tramadol, morphine, hydromorphone, oxycodone, diamorphone, alfentanil, allylprodine, alphaprodine, anileridine, benzylmorphine, benzitramide, buprenorphine, butorphanol, clonitazine, codeine, cyclazocin, desmorphine, dextromoramide, dezocine, dihydrocodeine, dihydromorphine, dimenoxadol, dimepheptanol, dimethylthiabutene, dioxaphetyl butyrate, dipipanone, eptazocine, ethoheptazine, levorphanol, methadone, meperidine, heroine or pharmaceutically acceptable salts thereof can be used. Considering from the viewpoint of pharmaceutics, the advantage of the preparations of the present invention can be achieved more effectively for drug of which daily dose is 10 mg or more and of which water-solubility is 1 mg/ml or more.

As said hydrophobic release-delaying additives, one or more ingredients selected from a group consisting of natural or synthetic waxes, fatty acids, fatty alcohols, fatty acid esters, fatty acid glycerides including mono-, di- and tri-glyceride, hydrocarbons, hydrogenated fats, hydrogenated castor oils and hydrogenated vegetable oils, can be used. Said fatty alcohols, through not particularly limited, include cetostearyl alcohol, stearyl alcohol, myristyl alcohol and lauryl alcohol, and said fatty acid esters, though not particularly limited, include glyceryl monostearate, glycerol monooleate, acetylated monoglyceride, tristearin, tripalmitin, cetyl ester wax, glyceryl palmitostearate and glyceryl behenate, and said wax, though not particularly limited, include beeswax, carnauba wax, glyco wax and castor wax. Said hydrophobic release-delaying additives act a role of surrounding drug uniformly, thus use of just small amount can effectively accomplish sustained-release property. As a hydrophobic release-delaying additive of the present invention, its melting point is preparably 30 to 150° C., more preferably 50 to 100° C.

As said hydrophobic wet-granulating material, at least one ingredient selected from a group consisting of fatty alcohols, fatty acids, fatty acid esters, fatty acid glycerides, preferably 50 to 100° C.

As said hydrophobic wet-granulating material, at least one ingredient selected from a group consisting of fatty alcohols, fatty acids, fatty acid esters, fatty acid glycerides, hydrocarbons, waxes, hydrogenated fats, hydrogenated castor oils, hydrogenated vegetable oils, alkyl cellulose and acrylic polymer can be used. Said hydrophobic wet-granulating material adheres to the surface of melt granules thereby to mask waxlike, surface property of melt granules, and to function secondary role in inducing release-delay.

In addition, the sustained-release preparations of the present invention can further comprise pharmaceutical additives such as diluents, binders, lubricants, etc. Said diluents, though not particularly limited, include lactose, dextrin, starch, micro-crystalline cellulose, calcium hydrogen phosphate, anhydrous calcium hydrogen phosphate, calcium carbonate, sugars, etc. Said binders, though not particularly limited, include polyvinylpyrrolidone, gelatin, starch, sucrose, methylcellulose, ethylcellulose, hydroxypropylcellulose, hydroxypropylalkylcellulose, etc. Said lubricants, though not particularly limited, include stearic acid, zinc stearate, magnesium stearate, calcium stearate, talc, etc.

In addition, the sustained-release preparations of the present invention can further comprise a coating layer including coating agent. Introduction of the coating layer enables easier control of drug release pattern. The drug release pattern can be controlled by thickness of coating layer. Additionally, for the control of drug release pattern, the coating layer can further comprise release-controlling materials. As said material, at least one selected from a group consisting of sugars, inorganic salts, organic salts, alkylcellulose, hydroxyalkylcellulose, hydroxypropylalkylcellulose, polyvinylpyrrolidone, polyvinylalcohol and drugs can be used. In case of sustained-release preparations to which coating layer is introduced, drug can be contained within the coating layer for rapid reaching effective blood level upon intake. Content of drug within coating layer is 1 to 50%, preferably 1 to 20% to total drug content of the preparation.

As said coating agent, at least one component selected from a group consisting of ethylcellulose, shellac, ammonio methacrylate copolymer, polyvinylacetate, polyvinylpyrrolidone, polyvinylalcohol, hydroxymethylcellulose, hydroxyethylcellulose, hydroxypropylcellulose, hydroxybutylcellulose, hydroxypentylcellulose, hydroxypropylmethylcellulose, hydroxypropylbutylcellulose, hydroxypropylpentylcellulose and Opadry (Colorcon Co.), can be used. As said ammonio methacrylate copolymer, for example, Eudragit RS™ or Eudragit RL™ can be used. Coating with coating agent can accomplish color endowment, stabilization, dissolution control and taste masking.

Said coating layer can further comprise plasticizer, and additionally include colors, antioxidant, talc, titanium dioxide, flavors, etc. As said plasticizer, one or more components selected from a group consisting of castor oil, fatty acids, substituted triglyceride and glyceride, polyethyleneglycol with molecular weight of 300 to 50,000 and its derivatives, can be used.

The present invention relates to preparation methods for sustained-release preparations of the present invention, comprising the following two steps:

(1) a drug is mixed with hydrophobic release-delaying additives and then the mixture is subjected to melt granulation thereby to prepare primary granules, and

(2) the granules obtained in step 1 are mixed with hydrophobic wet-granulating material and then the mixture is subjected to wet granulation thereby to prepare secondary granules.

This can be described in more detail as follows:

First, hydrophobic release-delaying additive is molten or softened by addition of energy (heat), followed by adding with drug and by mixing to homogeneity. The mixture is cooled below melting point or softening point of the hydrophobic release-delaying additives to form solid granules. The obtained granules are pulverized to uniform size and screened. Hydrophobic additives are added thereto and secondary wet-granulation process is carried cut thereby to prepare secondary granules. During the secondary wet-granulation process, pharmaceutical additives such as diluents, binders and lubricants can be further added. Said secondary granules can be filled into capsules, or compressed into tablets to prepare sustained-release preparations according to the present invention.

In addition, said preparation method can further comprise a step of coating the secondary granules or its compressed-granule into tablet with coating solution comprising coating agent. As solvent for the coating solution to form coating layer, water or organic solvent can be used, and it is preferred to use, as the organic solvent, methanol, ethanol, isopropanol, acetone, chloroform, dichloromethane or a mixture thereof.

### DESCRIPTION OF DRAWINGS

FIG. 1 shows a result of dissolution test for the sustained-release preparations prepared in Example 3 (▪), Example 6 (●), Example 13 (▴), Example 15 (□), and Comparative Example 2(?).

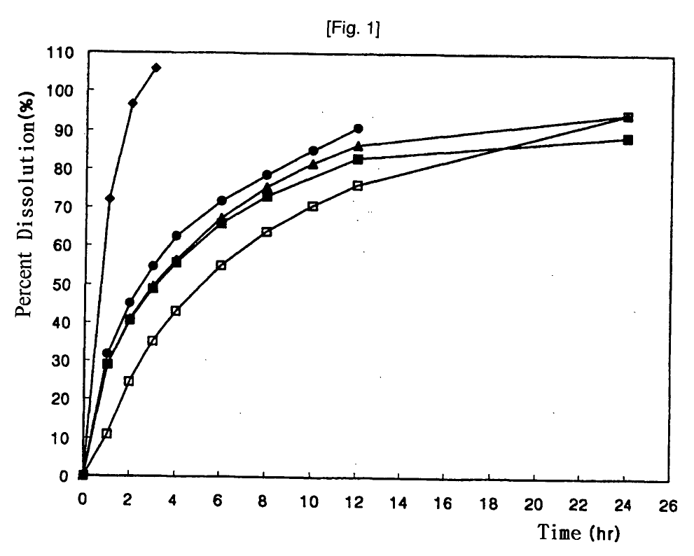

### BEST MODE

In the below, the present invention is explained in further detail through Examples or Experimental Examples. However, the scope of the present invention is not interpreted as being limited to the specific examples.

### MODE FOR INVENTION

### EXAMPLES 1 TO 3

### Preparation of Matrix Tablets Including Tramadol Hydrochloride

A mixture of glyceryl behenate and tramadol hydrochloride was heated to 70° C. with mixing until glyceryl behenate was melted or softened. The mixture was cooled to normal temperature to form solid mass. The mass was pulverized and passed through 20 mesh. The screened particles were mixed with the other additives listed in the following Table 1 and subjected to secondary wet-granulation. The prepared granules were dried, mixed with talc and magnesium stearate, and compressed into adequate form to prepare tablets. Composition of the obtained matrix tablet is represented in the following Table 1.

### COMPARATIVE EXAMPLE 1

Glyceryl behenate and tramadol hydrochloride were mixed and granules were prepared by passing through only melt granulation. Then, according to the same method as in the Example 1, tablets were prepared. Composition of the obtained matrix tablets is shown in the following Table 1.

### COMPARATIVE EXAMPLE 2

To the mixture of glyceryl behenate and tramadol hydrochloride, the other additives represented in the Table 1 were added and subjected to wet granulation, and then according to the same method as in Example 1, tablets were prepared. Composition of the obtained matrix tablets is shown in the following Table 1.

TABLE 1

| | | | | Compara- | Compara- |
|---|---|---|---|---|---|
| | | | | tive | tive |
| Ingredient | Example | Example | Example | Example | Example |
| (mg) | 1 | 2 | 3 | 1 | 2 |
| Tramadol | 150 | 150 | 150 | 150 | 150 |
| hydro- | | | | | |
| chloride | | | | | |
| Glyceryl | 85 | 95 | 120 | 120 | 30 |
| behenate | | | | | |
| Eudragit | 45 | 35 | 10 | — | 80 |
| RS PO | | | | | |
| Eudragit | — | — | — | — | 20 |
| RL PO | | | | | |
| Hydro- | — | — | 60 | — | — |
| genated | | | | | |
| castor | | | | | |
| oil | | | | | |
| Povidone | 17 | 17 | 3 | — | 17 |
| Talc | — | — | 3.5 | 14 | — |
| Magnesium | 3 | 3 | 3.5 | 6 | 3 |
| stearate | | | | | |
| Water* | q.s. | q.s. | q.s. | q.s. | q.s. |
| Total | 300 | 300 | 350 | 290 | 300 |

*removed during the process

### EXPERIMENTAL EXAMPLE 1

### Test for Effect on Surface Adhesion

Example 3 and Comparative Example 1 prepared the melt granules according to the same process by using same amount of melt granulating substance. In case of Example 3, adhesion property of the surface of the primary melt granules could be covered through secondary wet-granulation, thus adhesion toward punch or die was not observed during tablet process, while the granules prepared in Comparative Example 1 exhibited serious adhesion in spite of addition of excessive amount of lubricant, resulting in impossibility of tablet preparation.

### EXPERIMENTAL EXAMPLE 2

### Dissolution Test

Release tendencies of the matrix tablets prepared in the samples 1 to 3, and Comparative Example 2 were observed by using USP dissolution test device. Time-dependent dissolution of drug was determined under each test conditions of simulated intestinal solution (Solution II, pH 6.8) and paddle type II, 50 rpm/900 ml. The result is represented in the following Table 2.

TABLE 2

| | | | | Comparative |
|---|---|---|---|---|
| Time (hr) | Example 1 | Example 2 | Example 3 | Example 2 |
| 0 | 0.00 | 0.00 | 0.00 | 0.00 |
| 1 | 40.34 | 38.47 | 29.01 | 72.12 |
| 2 | 58.16 | 54.57 | 40.53 | 96.63 |
| 3 | 70.32 | 65.43 | 48.76 | 105.96 |
| 4 | 78.91 | 74.09 | 55.75 | — |
| 6 | 89.90 | 84.14 | 65.77 | — |
| 8 | 95.63 | 88.02 | 73.27 | — |
| 12 | 97.98 | 90.58 | 83.01 | — |
| 24 | 99.88 | 92.99 | 88.69 | — |

Based on the above result of dissolution test, it could be confirmed that, through melt granulation, effective drug-release delay was induced just by using relatively small amount of hydrophobic release-delaying additives. On the other hand, since surface adhesion of melt granules was covered via secondary wet-granulation, preparation of tablets was easy. The release rate could be controlled by the content of hydrophobic release-delaying additives.

### EXAMPLES 4 TO 6

### Preparation of Matrix Tablets Including Tramadol Hydrochloride

A mixture of hydrogenated castor oil and tramadol hydrochloride was heated to 75° C. with mixing until hydrogenated castor oil was melted or softened. This was cooled to normal temperature to form solid mass. The mass was pulverized and screened with 20 mesh. Particles passed through the mesh were mixed with the other additives listed in the Table 3 and subjected to secondary wet-granulation. The prepared granules were dried, mixed with magnesium stearate, and compressed into adequate form to prepare tablets. Composition of the matrix tablets is given in the following Table 3.

TABLE 3

| Ingredient (mg) | Example 4 | Example 5 | Example 6 |
|---|---|---|---|
| Tramadol hy- | 150 | 150 | 150 |
| drochloride | | | |
| Hydrogenated castor | 70 | 80 | 100 |
| oil | | | |
| Eudragit RS PO | 47 | 37 | 37 |
| Povidone | 30 | 30 | 10 |
| Magnesium stearate | 3 | 3 | 3 |
| Water* | q.s. | q.s. | q.s. |
| Total | 300 | 300 | 300 |

*removed during the process

### EXPERIMENTAL EXAMPLE 3

### Dissolution Test

Time-dependent dissolutions of drug from the coated matrix tablets prepared in Examples 4 to 6 were determined according to the same method as in Experimental Example 2. The result is shown in the following Table 4.

TABLE 4

| Time (hr) | Example 4 | Example 5 | Example 6 |
|---|---|---|---|
| 0 | 0.00 | 0.00 | 0.00 |
| 1 | 45.08 | 43.06 | 31.66 |
| 2 | 61.56 | 56.41 | 45.05 |
| 3 | 74.32 | 65.93 | 54.65 |
| 4 | 82.20 | 72.31 | 62.52 |
| 6 | 91.00 | 82.45 | 71.70 |
| 8 | 94.00 | 87.15 | 78.50 |
| 10 | 97.85 | 93.38 | 82.14 |
| 12 | 99.01 | 98.05 | 90.74 |

From the above result of dissolution test, it could be confirmed that release rate can be controlled with the content of hydrophobic release-delaying additives.

### EXAMPLES 7 AND 8

### Coating of Matrix Tablets Containing Tramadol Hydrochloride

The matrix tablets prepared in said Example 3 were coated with acrylic polymer mixture. The tablets were subjected to spray-coating in coating pan with coating solution of composition shown in the Table 5, and dried in an oven at 40 to 50° C. for 12 to 24 hrs.

TABLE 5

| Composition of coating | | |
|---|---|---|
| solution (%) | Example 7 | Example 8 |
| Eudragit RS 100 | 2.48 | 3.34 |
| Eudragit RL 100 | 3.30 | 1.66 |
| Polyethyleneglycol 4,000 | 0.50 | 0.50 |
| Talc | 2.48 | 2.50 |
| Water | 0.99 | 1.00 |
| Acetone | 41.65 | 42.00 |
| Isopropanol | 48.60 | 49.00 |
| Coating %* | 3 | 3 |

*coating ratio to uncoated core-matrix tablets was represented by weight %.

### EXPERIMENTAL EXAMPLE 4

### Dissolution Test

Time-dependent dissolutions of drug from the coated matrix tablets prepared in Examples 7 and 8 were determined by the same method as in Experimental Example 2. The result is represented in the following Table 6.

TABLE 6

| Time (hr) | Example 7 | Example 8 |
|---|---|---|
| 0 | 0.00 | 0.00 |
| 1 | 22.22 | 14.62 |
| 2 | 35.28 | 26.90 |
| 3 | 42.68 | 35.21 |
| 4 | 50.07 | 42.00 |
| 6 | 60.66 | 52.08 |
| 8 | 68.54 | 59.80 |
| 10 | 75.21 | 65.93 |
| 12 | 79.75 | 71.01 |
| 24 | 95.90 | 88.32 |

From the above result of dissolution test, it could be confirmed that ultimate drug-release pattern can be controlled by regulating the relative ratio of the two substances (Eudragit RS 100 and RL 100) which form the coating layer and differ in permeability to water.

### EXAMPLES 9 TO 11

### Coating of Matrix Tablet Including Tramadol Hydrochloride

The matrix tablets prepared in the Example 3 were coated with a mixture of ethyl-cellulose and hydroxypropylmethylcellulose. The tablets were subjected to spray-coating in coating pan with coating solution of composition shown in the following Table 7, and then dried in an oven at 40 to 50° C. for 12 to 24 hrs.

TABLE 7

| Composition of | | | |
|---|---|---|---|
| coating solution (%) | Example 9 | Example 10 | Example 11 |
| Ethylcellulose | 3.6 | 4.2 | 5.4 |
| Hydroxypropyl- | 2.4 | 1.8 | 0.6 |
| methylcellulose | | | |
| Castor oil | 0.6 | 0.6 | 0.6 |
| Ethanol | 46.7 | 46.7 | 46.7 |
| Methylenechloride | 46.7 | 46.7 | 46.7 |
| Coating %* | 8 | 8 | 8 |

*coating ratio to uncoated core matrix tablets was represented by weight %

### EXPERIMENTAL EXAMPLE 5

### Dissolution Test

Time-dependent dissolutions of drug from the coated matrix tablets prepared in the Examples 9 to 11 were determined by the same method as in Experimental Example 2. The result is represented in the following Table 8.

TABLE 8

| Time (hr) | Example 9 | Example 10 | Example 11 |
|---|---|---|---|
| 0 | 0.00 | 0.00 | 0.00 |
| 1 | 22.63 | 13.92 | 4.16 |
| 2 | 34.44 | 26.74 | 7.85 |
| 3 | 42.48 | 35.52 | 11.64 |
| 4 | 49.56 | 42.21 | 15.27 |
| 6 | 59.02 | 52.52 | 21.57 |
| 8 | 66.61 | 60.10 | 27.38 |
| 10 | 73.37 | 63.32 | 32.60 |
| 12 | 78.64 | 67.65 | 37.29 |
| 18 | 89.56 | 78.20 | 49.32 |
| 24 | 95.13 | 84.38 | 60.02 |

From the above result of dissolution test, it could be confirmed that ultimate release pattern of drug can be controlled by regulating the relative ratio of the two substances forming the coating layer and differing in water-solubility.

### EXAMPLES 12 AND 13

### Preparation of Matrix Tablets Containing Tramadol Hydrochloride

A mixture of hydrogenated castor oil and tramadol hydrochloride was heated to 75° C. with mixing until hydrogenated castor oil softened. Then this was cooled to normal temperature to form solid mass. The mass was pulverized and screened with 20 mesh. Particles passed thrash the mesh were mixed with the additives listed in the following Table 9 and subjected to secondary wet-granulation. Thus prepared granules were dried, mixed with magnesium stearate, and then compressed to adequate form to prepare tablets. Composition of the matrix tablets is given in the following Table 9.

TABLE 9

| Ingredient (mg) | Example 12 | Example 13 |
|---|---|---|
| Tramadol hydrochloride | 150 | 150 |
| Hydrogenated castor oil | 150 | 150 |
| Ethylcellulose | 62 | 62.2 |
| Povidone | 0.2 | — |
| Talc | 10.2 | 10.2 |
| Magnesium stearate | 7.6 | 7.6 |
| Ethanol* | q.s. | q.s. |
| Total | 380 | 380 |

*removed during the process

### EXPERIMENTAL EXAMPLE 6

### Dissolution Test

Time-dependent dissolutions of drug from the coated matrix tablets prepared in the Examples 12 and 13 were determined by the same method as in Experimental Example 2. The result is represented in the following Table 10.

TABLE 10

| Time (hr) | Example 12 | Example 13 |
|---|---|---|
| 0 | 0.00 | 0.00 |
| 1 | 28.26 | 28.99 |
| 2 | 39.49 | 40.90 |
| 3 | 47.83 | 49.43 |
| 4 | 54.57 | 56.33 |
| 6 | 65.59 | 67.29 |
| 8 | 74.26 | 75.40 |
| 10 | 80.71 | 81.68 |
| 12 | 85.92 | 86.39 |
| 24 | 97.46 | 94.59 |

### EXAMPLES 14 AND 15

### Coating of Matrix Tablet Containing Tramadol Hydrochloride

The matrix tablets prepared in the Examples 12 and 13 were coated separately with a mixture of ethylcellulose and hydroxypropylmethylcellulose. The tablets were subjected to spray coating in coating pan with coating solution of composition shown in the following Table 11, and then dried in an oven at 40 to 50° C. for 12 to 24 hrs.

TABLE 11

| Composition of coating | | |
|---|---|---|
| solution (%) | Example 14 | Example 15 |
| Ethylcellulose | 4.0 | 4.0 |
| Hydroxypropyl methyl- | 1.7 | 1.7 |
| cellulose | | |
| Castor oil | 0.5 | 0.5 |
| Ethanol | 35.4 | 35.4 |
| Methylenechloride | 58.4 | 58.4 |
| Coating %* | 6 | 6 |

*coating ratio to uncoated core matrix tablets was represented by weight %

### EXPERIMENTAL EXAMPLE 7

### Dissolution Test

Time-dependent dissolutions of drug from the matrix tablets prepared in Examples 14 and 15 were determined by the same method as in Experimental Example 2. The result is represented in the following Table 12.

TABLE 12

| Time (hr) | Example 14 | Example 15 |
|---|---|---|
| 0 | 0.00 | 0.00 |
| 1 | 13.95 | 10.78 |
| 2 | 27.19 | 24.45 |
| 3 | 36.19 | 35.14 |
| 4 | 43.27 | 42.97 |
| 6 | 54.54 | 54.99 |
| 8 | 63.27 | 63.79 |
| 10 | 70.10 | 70.84 |
| 12 | 75.66 | 76.16 |
| 24 | 91.62 | 94.68 |

From the above result of dissolution test, it could be confirmed that sustained-release preparations, which exhibit sustained-release of drug over 24 hrs, can be obtained according to the present invention.

### EXAMPLES 16˜21

### Coating of Matrix Tablet Containing Tramadol Hydrochloride

The matrix tablets prepared in the Examples 13 were coated separately with a mixture of ethylcellulose and hydroxypropylmethylcellulose. The tablets were subjected to spray coating in H-coater with coating solution of composition shown in the following Table 13.

TABLE 13

| Composition of | | | | | | |
|---|---|---|---|---|---|---|
| coating | Example | Example | Example | Example | Example | Example |
| solution (%) | 16 | 17 | 18 | 19 | 20 | 21 |
| Ethylcellulose | 5.03 | 5.06 | 5.08 | 5.10 | 5.10 | 5.10 |
| Hydroxypropyl | 2.71 | 2.17 | 1.69 | 1.28 | 1.28 | 1.28 |
| methylcellulose | | | | | | |
| Castor oil | 0.77 | 0.78 | 0.78 | 0.78 | 0.78 | 0.78 |
| Ethanol | 73.19 | 73.60 | 73.96 | 74.27 | 74.27 | 74.27 |
| Purified water | 18.30 | 18.40 | 18.49 | 18.57 | 18.57 | 18.57 |
| Coating %* | 6 | 5.6 | 5.27 | 1 | 2 | 3 |

*coating ratio to uncoated core matrix tablets was represented by weight %

### EXPERIMENTAL EXAMPLE 8

### Dissolution Test

Release tendencies of the matrix tablets prepared in the Examples 16 to 21, and Example 13 were observed by using USP dissolution test device. Time-dependent dissolution of drug was determined under each test conditions of water and paddle type II, 100 rpm/900 ml. The result is represented in the following Table 14

TABLE 14

| | Example | Example | Example | Example | Example | Example | Example |
|---|---|---|---|---|---|---|---|
| [symbol] (hr) | 13 | 16 | 17 | 18 | 19 | 20 | 21 |
| 0 | 0.00 | 0.00 | 0.00 | 0.00 | 0.00 | 0.00 | 0.00 |
| 1 | 34.10 | 15.89 | 6.42 | 0.52 | 19.22 | 3.18 | 0.44 |
| 3 | 55.02 | 37.55 | 35.44 | 1.02 | 42.05 | 24.25 | 9.27 |
| 7 | 76.46 | 60.04 | 65.71 | 3.60 | 68.16 | 50.84 | 34.64 |
| 19 | 96.83 | 88.35 | 94.90 | 14.64 | 103.43 | 101.97 | 80.24 |
| 24 | 97.78 | 103.11 | 96.97 | 20.19 | | | 100.09 |

From the above result of dissolution test, it could be confirmed that, according to the present invention, the release pattern of drug from sustained-release preparations can be controlled via introducing of coating layer into uncoated matrix tablet such as the preparation of Example 13.

From the results of Example 16 to 18, in which hydroxypropyl methylcellulose was used as a release-controlling material, it could be confirmed that dissolution of drug can be controlled according to the content of the release-controlling material. Especially, the release pattern of drug was controlled by controlling of the ratio of hydroxypropyl methylcellulose, a hydrophilic release-controlling material, to ethylcellulose, a hydrophobic coating agent. It is because flux of external fluid into inside of matrix tablets is controlled by size and number of pores formed in coating layer due to dissolving of release-controlling material.

From the results of Example 19 to 21, in which the ratio of a hydrophilic release-controlling material to a hydrophobic coating agent was fixed, it could be confirmed that release pattern of drug can be controlled according to the thickness of the coating layer.

### INDUSTRIAL APPLICABILITY

The sustained-release preparations according to the present invention enables maintenance of effective blood concentration of drug for many hours via sustained release of the drug over 12 hours or more, and further its production is easy owing to convenience of process.

1. Sustained-release preparations characterized by being prepared from double granules which are obtained by primary granulation of drug according to melt granulation using hydrophobic release-delaying additives, and then by secondary granulation of the obtained granules according to wet granulation using hydrophobic wet-granulation material.

2. The sustained-release preparations in claim 1, characterized in containing 0.5 to 80% by weight of drug, 10 to 65% by weight of hydrophobic release-delaying additive, and 1 to 35% by weight of hydrophobic wet-granulation material.

3. The sustained-release preparations in claim 1 or 2, characterized in that said drug is tramadol, morphine, hydromorphone, oxycodone, diamorphone, alfentanil, allylprodine, alphaprodine, anileridine, benzylmorphine, benzitramide, buprenorphine, butorphanol, clonitazine, codeine, cyclazocin, desmorphine, dextromoramide, dezocine, dihydrocodeine, dihydromorphine, dimenoxadol, dimepheptanol, dimethylthiabutene, dioxaphetyl butyrate, dipipanone, eptazocine, ethoheptazine, levorphanol, methadone, meperidine, heroine or pharmaceutically acceptable salts thereof.

4. The sustained-release preparations in claim 1 or 2, characterized in that said hydrophobic release-delaying additive is one or more ingredients selected from a group consisting of natural or synthetic waxes, fatty acids, fatty alcohols, fatty acid esters, fatty acid glycerides including mono-, di- and tri-glyceride, hydrocarbons, hydrogenated fats, hydrogenated castor oils and hydrogenated vegetable oils.

5. The sustained-release preparations in claim 4, characterized in that said fatty alcohols are one or more ingredients selected from a group consisting of cetostearyl alcohol, stearyl alcohol, myristyl alcohol and lauryl alcohol, and said fatty acid esters are one or more ingredients selected from a group consisting of glyceryl monostearate, glycerol monooleate, acetylated monoglyceride, tristearin, tripalmitin, cetyl ester wax, glyceryl palmitostearate and glyceryl behenate, and said waxes are one or more ingredients selected from a group consisting of beeswax, carnauba wax, glyco wax and castor wax.

6. The sustained-release preparations in claim 1 or 2, characterized in that said hydrophobic wet-granulating materials are one or more ingredients selected from a group consisting of fatty alcohols, fatty acids, fatty acid esters, fatty acid glycerides, hydrocarbons, waxes, hydrogenated fats, hydrogenated castor oils, hydrogenated vegetable oils, alkyl cellulose and acrylic polymer.

7. The sustained-release preparations in claim 1 or 2, characterized in further comprising pharmaceutical additives such as diluents, binders and lubricants.

8. The sustained-release preparations in claim 1 or 2, characterized in further containing a coating layer including coating agent.

9. The sustained-release preparations in claim 8, characterized in that the coating layer further comprise release-controlling materials, said material is at least one selected from a group consisting of sugars, inorganic salts, organic salts, alkylcellulose, hydroxyalkylcellulose, hydroxypropylalkylcellulose, polyvinylpyrrolidone, polyvinylalcohol and drugs.

10. The sustained-release preparations in claim 8, characterized in that said coating layer contains drug of 1 to 50% to total drug content of the preparation.

11. The sustained-release preparations in claim 8, characterized in that said coating agent is one or more component selected from a group consisting of ethylcellulose, shellac, ammonio methacrylate copolymer, polyvinylacetate, polyvinylpyrrolidone, polyvinylalcohol, hydroxymethylcellulose, hydroxyethylcellulose, hydroxypropylcellulose, hydroxybutylcellulose, hydroxypentylcellulose, hydroxypropylmethylcellulose, hydroxypropylbutylcellulose and hydroxypropylpentylcellulose.

12. A method for preparing the sustained-release preparations of claim 1, comprising (1) a drug is mixed with hydrophobic release-delaying additives and subjected to melt granulation thereby to prepare primary granules, and (2) thus obtained granules are mixed with hydrophobic wet-granulating material and subjected to wet granulation thereby to prepare secondary granules.

13. The method in claim 12, characterized in further comprising a step of coating said secondary granules or its compressed-granules into tablet with coating solution comprising coating agent.
